(19) United States
(12) Patent Application Publication
(10) Pub. No.: US 2023/0053748 A1
(43) Pub. Date: Feb. 23, 2023

(54) MICROBIAL COMBINATIONS AND USES THEREOF

(71) Applicant: BUZZELET DEVELOPMENT AND TECHNOLOGIES LTD., OR-AKIVA (IL)

(72) Inventors: AHARON EYAL, JERUSALEM (IL); NOA RAZ, GIZO (IL)

(21) Appl. No.: 17/880,923

(22) Filed: Aug. 4, 2022

Related U.S. Application Data
(63) Continuation-in-part of application No. PCT/IB2021/050893, filed on Feb. 4, 2021.
(60) Provisional application No. 62/975,223, filed on Feb. 12, 2020.
(60) Provisional application No. 62/970,721, filed on Feb. 6, 2020.

(51) Int. Cl. A61K 35/741; A23L 33/135

(52) U.S. Cl. CPC A61K 35/741; A23L 33/135

(57) ABSTRACT

A method of treating a condition and/or a symptom associated with an effect of at least one element of the endocannabinoid system in an animal, the method including administering to the animal (i) a therapeutically effective amount of at least one microorganism and/or of a prebiotic thereof, where the microorganism is capable of modulating an effect of at least one element of the endocannabinoid system of the animal and (ii) a therapeutically effective amount of at least one compound capable of modulating an effect of at least one element of the endocannabinoid system of the animal. Further provided are compositions for use in such methods.

### CROSS-REFERENCE TO RELATED APPLICATIONS

The present application is a Continuation-in-Part of International Application No. PCT/IB2021/050893, filed Feb. 4, 2021, which claims priority from U.S. Provisional Patent Application No. 62/970,721, filed 6 Feb. 2020, and U.S. Provisional Patent Application No. 62/975,223, filed 12 Feb. 2020, both of which are incorporated by reference as if fully set-fort herein.

### FIELD OF THE INVENTION

The field of art to which this invention generally pertains is microbial compositions, and more specifically to compositions comprising a microorganism and/or a prebiotic thereof capable of modulating at least one element of the endocannabinoid system and uses thereof in therapy.

### BACKGROUND OF THE INVENTION

Cannabis is a complex plant comprising over 400 chemical entities of which more than 60 are cannabinoid compounds, some of which have opposing pharmacological effects. In the naturally occurring Cannabis plant, cannabinoids are synthesized and accumulated as cannabinoid acids (e.g. tetrahydrocannabinolic acid (THCa) and cannabidioolic acid (CBDA). When the herbal product is dried, stored and heated, the acids decarboxylize into their active forms, such as tetrahydrocannabinol (THC) and Cannabidiol (CBD) [De Meijer et al. 2003; The inheritance of chemical phenotype in Cannabis sativa L. Genetics 63: 335-346]. There are major differences in functional pharmaceutical effects between the acid and decarboxylized forms (for instance THC is psychoactive, while THCa is not psychoactive), in physical properties (such as solubility and boiling point) and in chemical properties (e.g. THCa may undergo esterification and dimerization, while THC cannot, THCa solubility is pH dependent, while that of THC is not, etc.) (e.g. De Zeeuw, Journal of Pharmacy and Pharmacology, 1972; Mechoulam, Naturwissenschaften, 1978; Grotenhermen, Clinical Pharmacokinetics, 2003).

The endocannabinoid system (ECS) is a neuromodulatory system that plays major roles in central nervous system development, synaptic plasticity and the response to endogenous and environmental insults. The ECS comprises endocannabinoids, which are endogenous lipid-based neurotransmitters; cannabinoid receptors which are expressed throughout the central and peripheral nervous systems of vertebrates; and enzymes responsible for the synthesis and degradation of the endocannabinoids.

### SUMMARY OF THE INVENTION

According to an aspect of the present invention, there is provided a method of treating a condition and/or a symptom associated with an effect of at least one element of the endocannabinoid system in an animal, the method comprising administering to said animal (i) a therapeutically effective amount of at least one microorganism and/or of a prebiotic thereof, wherein said microorganism is capable of modulating an effect of at least one element of the endocannabinoid system of said animal and (ii) a therapeutically effective amount of at least one compound capable of modulating an effect of at least one element of the endocannabinoid system of said animal.

According to a further aspect of some embodiments of the present invention, there is provided a composition comprising (i) a therapeutically effective amount of at least one microorganism and/or of a prebiotic thereof, wherein said microorganism is capable of modulating an effect of at least one element of the endocannabinoid system, and (ii) a therapeutically effective amount of at least one compound is capable of modulating an effect of at least one element of the endocannabinoid system.

### DETAILED DESCRIPTION OF THE INVENTION

The present invention relates to combinations of a microorganism and/or a prebiotic thereof with at least one compound and uses thereof in therapy.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

Unless indicated otherwise, the term “cannabinoid” as used herein refers to a compound that affects the endocannabinoid system. Cannabinoids are agonists or antagonists to receptors in the endocannabinoid system. Exogenous cannabinoids originating from the cannabis plant (also referred to as phytocannabinoids), may alter the availability of endogenous cannabinoids to the cannabinoid receptors.

As used herein, the term “prebiotic” refers to a substrate that is selectively utilized by particular microorganisms conferring a health benefit.

As used herein, the term “probiotic” refers to a live microorganism that, when administered in adequate amounts, confers a health benefit on the host

As used herein, the term “modulating an effect” refers to altering, such as increasing or decreasing a physiological effect of an element.

For the avoidance of doubt, it is to be understood that the at least one compound is not a microorganism, a prebiotic or a portion thereof, such that the microorganism. and/or prebiotic is different from the at least one compound used in the methods and compositions disclosed herein.

According to some embodiments, the microorganism and the at least one compound modulate the same element. According to some embodiments, the microorganism and the at least one compound have the same modulatory effect on the same element e.g. both increase the activity and/or expression of the same element or both decrease the activity and/or expression of the same element. According to some embodiments, the microorganism and the at least one compound have different modulatory effects on the same element e.g the microorganism increases the activity and/or expression of the same element and the at least one compound decreases the activity and/or expression of the same element, or vice versa.

According to some embodiment, the microorganism and the at least one compound modulate different elements.

According to some embodiments, the microorganism and the at least one compound modulate the same element, wherein said same element refers to the same particular element. According to some embodiments, the microorganism and the at least one compound modulate the same element, wherein said same element refers to the same category of elements (e.g. the group of cannabinoid receptors, the groups of endocannabionids). According to some embodiments, modulating an effect is achieved by modulating (by increasing or decreasing) expression of an element. According to some embodiments, modulation is achieved by increasing or decreasing a physiological effect of an element.

As used herein, the term “direct modulation” refers to directly acting on an element of the endocannabinoid system in order to modulate an effect of that element.

As used herein, the term “indirect modulation” refers to indirectly acting on an element of the endocannabinoid system in order to modulate an effect of that element, e.g. by reducing or increasing expression of the element.

As used herein, the term “animal” refers to any animal, including a human being.

As used herein, the term “neuromodulator” refers to is a compound released from a neuron that affects groups of neurons, or effector cells that have the appropriate receptors.

As used herein, the term “THC” refers to THCa (tetrahydrocannabiniolic acid) and/or to THC (tetrahydrocannabiniol) unless indicated otherwise. As used herein, the term “CBD” refers to CBDa (cannabidiolic acid) and/or to CBD (cannabidiol) unless indicated otherwise. Thus, the term “CBD to THC ratio” may mean “CBD to THC ratio”, “CBDa to THC ratio”, “CBD to THCa ratio”, “CBDa to THCa ratio”, “CBD to THC+THCa ratio”, “CBDa to THC+THCa ratio”, “CBD+CBDa to THC ratio”, “CBD+CBDa to THCa ratio” or “CBD+CBDa to THC+THCa ratio”. As used herein, the term “CBG” refers to CBGa (cannabigerolic acid) and/or to CBG (cannabigerol) unless indicated otherwise. As used herein, the term “CBN” refers to CBNa (Cannabinolic acid) and/or to CBN (cannabinol) unless indicated otherwise. As used herein, the term “CBC” refers to CBCa (cannabichromenic acid) and/or to CBC (Cannabichromene) unless indicated otherwise. As used herein, the term “CBL” refers to CBLa (Cannabicycol acid) and/or to CBL (Cannabicyclol) unless indicated otherwise. As used herein, the term “THCV” refers to THCVa (tetrahydrocannabivarin acid) and/or to THCV (tetrahydrocannabivarin) unless indicated otherwise. As used herein, the term “CBDV” refers to CBDVa (cannabigerovarin acid) and/or to CBDV (cannabidivarin) unless indicated otherwise.

As used herein, the term “element of the endocannabinoid system” refers to an endocannabinoid, a cannabinoid receptor and an enzyme responsible for the synthesis or degradation of an endocannabinoid.

As used herein, the term “treating” includes ameliorating, mitigating, and reducing the instances of a disease or condition, or the symptoms of a disease or condition.

As used herein, the term “administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, intramuscular, intraosseous, intrathecal, and transdermal, or combinations thereof. “Administering” can also include providing a different compound that when ingested or delivered as above will necessarily transform into the compound that is desired to be administered, this type of “different compound” is often being referred to as a “Prodrug”.

As used herein, the term “therapeutically effective amount” means the amount of an active substance that, when administered to a subject for treating a disease, disorder, or other undesirable medical condition, is sufficient to have a beneficial effect with respect to that disease, disorder, or condition. The therapeutically effective amount will vary depending on the chemical identity and formulation form of the active substance, the disease or condition and its severity, and the age, weight, and other relevant characteristics of the patient to be treated. Determining the therapeutically effective amount of a given active substance is within the ordinary skill of the art and typically requires no more than routine experimentation.

Unless indicated otherwise, percent is weight percent and ratio is weight/weight ratio. Unless indicated otherwise, weight ratio means the ratio between weight content, e.g. in an aqueous solution containing 20% solute and 80% water, the solute to water weight ratio is 20:80 or 1:4.

As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Aspects and embodiments of the invention are described in the specification hereinbelow and in the appended claims.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, there is provided a method of treating a condition and/or a symptom associated with an effect of at least one element of the endocannabinoid system in an animal, the method comprising administering to said animal (i) a therapeutically effective amount of at least one microorganism and/or of a prebiotic thereof, wherein said microorganism is capable of modulating an effect of at least one element of the endocannabinoid system of said animal and (ii) a therapeutically effective amount of at least one compound capable of modulating an effect of at least one element of the endocannabinoid system of said animal.

According to an aspect of some embodiments of the present invention, there is provided (i) a therapeutically effective amount of at least one microorganism and/or of a prebiotic thereof, wherein said microorganism is capable of modulating an effect of at least one element of the endocannabinoid system of said animal and (ii) a therapeutically effective amount of at least one compound capable of modulating an effect of at least one element of the endocannabinoid system of said animal, for use in therapy.

According to an embodiment, the at least one compound is capable of modulating said element, in some embodiments by direct modulation and in some embodiments by indirect modulation. According to one embodiment, said at least one microorganism is already present in the animal prior to administration. According to one such embodiment wherein said at least microorganism is already present in the animal, administration of said organism provides a further amount of said microorganism to said animal. According to one such embodiment wherein said at least microorganism is already present in the animal, a prebiotic thereof is administered, thereby facilitating growth of the microorganism. According to one embodiment, said at least one microorganism is not present in the animal prior to administration.

According to an embodiment, the at least one compound comprises at least one compound, at least two compound, at least three, at least four or at least five compound. According to an embodiment, the content of each compound is at least 10 parts per million (ppm).

According to some embodiments, the compound is a cannabinoid. As known in the art, cannabinoids have an acid form and a non-acid form (which is also referred to as decarboxylated form, since it can be generated by decarboxylating the acid form). The acid form is indicated herein by the letter (a) at the end of the cannabinoid acronym, e.g. tetrahydrocannabiniolic acid is indicated as THCa, while the decarboxylated form is THC. According to an embodiment, the cannabinoids are selected from the group consisting of tetrahydrocannabiniol in acid or decarboxylated form (THCa or THC, respectively), cannabidiol in acid or decarboxylated form (CBDa or CBD, respectively), cannabigerol in acid or decarboxylated form (CBGa or CBG, respectively), cannabichromene in acid or decarboxylated form (CBCa or CBC, respectively) tetrahydrocannabivarin in acid or decarboxylated form (THCVa or THCV, respectively), Cannabidivarin in acid or decarboxylated form (CBDVa or CBDV respectively) and cannabinol in acid or decarboxylated form (CBNa or CBN, respectively), Cannabicyclol in acid or decarboxylated form (CBLa or CBL, respectively).

According to an embodiment, at least one of the cannabinoids is in acid form. According to an embodiment, at least one of the cannabinoids is at least partially in decarboxylated form. According to an embodiment, at least 50% of the cannabinoids is in decarboxylated form, at least 60%, at least 70%, at least 80% or at least 90%.

According to an embodiment, the at least one cannabinoid comprises THC and/or THCa. According to an embodiment, the at least one cannabinoid comprises CBD and/or CBDa. According to an embodiment, the at least one cannabinoid comprises THC and/or THCa at a content of less than 1%, less than 0.8%, less than 0.6%, less than 0.4% or less than 0.2%. According to an embodiment, the at least one cannabinoid comprises both CBD and/or CBDa and THC and/or THCa and the weight/weight ratio between CBD and/or CBDa and THC and/or THCa ((CBD+CBDa)/(THC+THCa)) is at least 10, at least 15, at least 20, at least 25 or at least 30. According to an embodiment, the at least one cannabinoid comprises CBG and/or CBGa. According to an embodiment, the at least one cannabinoid comprises CBN and/or CBNa. According to an embodiment, the at least one cannabinoid comprises CBC and/or CBCa. According to an embodiment, the at least one cannabinoid comprises CBL and/or CBLa. According to an embodiment, the at least one cannabinoid comprises THCV and/or THCVa. According to an embodiment, the at least one cannabinoid comprises CBDV and/or CBDVa.

According to an embodiment, said condition and/or symptom is associated with at least one selected from the group consisting of the gastrointestinal tract, adipose tissue, and the immune system.

According to an embodiment, said condition and/or symptom is associated with autism spectrum disorder.

According to an embodiment, said condition and/or symptom associated autism spectrum disorder is selected from the group consisting of of restlessness, rage attack, agitation, treatment-resistant self-injurious behavior, stress, aggression, anxiety, irritability, behavioral outbreaks, communication problems, disruptive behavior, psychosis, hyperactivity, lethargy, seizure activity, hyper sensitivity, stereotypy, inappropriate speech, sleep problem, cognitive impairment, motor and/or vocal tics, digestive problems, lack of appetite, constipation, depression, incontinence, difficulty in concentration, attention disorder and combinations thereof.

According to an embodiment, said condition and/or symptom is associated with a clinical deficiency of an endocannabinoid.

According to an embodiment, said condition and/or symptom is selected from the group consisting of migraine, fibromyalgia, irritable bowel diseases, diabetes, post-traumatic stress disorders, affective disorders, anorexia, motion sickness, multiple sclersosis, Parkinson disease and combinations thereof.

According to an embodiment, said element is expressed in the gastrointestinal tract.

According to an embodiment, said element of the endocannabinoid system is selected from the group consisting of endocannabinoid receptors, endocannabinoids, endocannabinoids synthesizing enzymes, endocannabinoids degrading enzymes, and combinations thereof.

According to an embodiment, said element is selected from the group consisting of CB1 receptors, CB2 receptors, TRP channels, GPR 18 receptors, GPR55 receptors, GPR119 receptor, Peroxisome proliferator-activated receptors (PPARs), 5-HT receptors, N-arachidonoylethanolamine (Anandamide), 2-arachidonoylglycerol (2-AG), phospholipase A2, phospholipase C, N-acetylphosphatidylethanolamine-hydrolysing phospholipase D (NAPE-PLD), diacylglycerol lipases (DAGs), fatty acid amide hydrolase (FAAH), monoacylglycerol lipase (MAGL) and combinations thereof.

According to an embodiment, said modulating an effect of at least one element comprises modulating the expression of at least one element selected from the group consisting of endocannabinoid receptors, endocannabinoids, endocannabinoids synthesizing enzymes, endocannabinoids degrading enzymes and combinations thereof.

According to an embodiment, said modulating an effect of at least one element comprises modulating the activity of at least one element selected from the group consisting of endocannabinoid receptors, endocannabinoids, endocannabinoids synthesizing enzymes, endocannabinoids degrading enzymes and combinations thereof.

According to an embodiment, said modulating an effect of at least one element comprises activating cannabinoid receptors, inactivating cannabinoid receptors, modulating the expression of endocannabinoids, modulating the expression of endocannabinoids synthesizing enzymes, modulating the expression of endocannabinoids degrading enzymes, modulating the action of another cannabinoid at the cannabinoid receptors and combinations thereof.

According to an embodiment, said microorganism changes the expression of cannabinoid type 1 (CB1) receptors and/or cannabinoid type 1 (CB2) receptors.

According to an embodiment, said microorganism changes the expression of transient receptor potential (TRP) channels.

According to an embodiment, said microorganism changes the expression of Peroxisome proliferator-activated receptors (PPARs).

According to an embodiment, said microorganism changes the expression of anandamide and/or the expression of 2-Arachidonoylglycerol (2AG).

According to an embodiment, said microorganism changes the expression of fatty acid amide hydrolase (FAAH) and/or the expression of N-acyl phosphatidylethanolamine phospholipase D (NAPE-PLD).

According to an embodiment, said microorganism changes the expression of monoacylglycerol lipase (MAGL) and/or the expression of diacylglycerol (DAG) lipases.

According to an embodiment, said at least one compound is an agonist of a gastrointestinal tract endocannabinoid receptor.

According to an embodiment, said at least one compound is an antagonist of a gastrointestinal tract endocannabinoid receptor.

According to an embodiment, said at least one compound is an allocentric modulator of a gastrointestinal tract endocannabinoid receptor.

According to an embodiment, said at least one compound changes the expression of anandamide, the expression of 2AG or both.

According to an embodiment, said at least one compound changes the expression of FAAH, the expression of NAPE-PLD, or both.

According to an embodiment, said at least one compound changes the expression of MAGL, the expression of DAG lipases, or both.

According to an embodiment, said at least one compound changes the expression of at least one selected from the group of CB1 receptors, CB2 receptors, TRP channels, Peroxisome proliferator-activated receptors (PPARs), and combination thereof.

According to an embodiment, said at least one compound is a cannabinoid.

According to an embodiment, said at least one compound is a phytocannabinoid and/or a synthetic cannabinoid.

According to an embodiment, said cannabinoid is selected from the group consisting of CBD, CBDA, CBDV, THCV, THC, THCA, CBG, CBGV, CBC, CBN and combination thereof.

According to an embodiment, said at least one compound is not a cannabinoid.

According to an embodiment, wherein said at least one compound is a terpene. According to one such embodiment, said terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, geranyl acetate, camphor, menthol, iso-menthone, neral, gerial, viridiflorol, germacrene, thymol, Menth-2-en-1-ol, farensol, carotol, myrtenol, citral and combination thereof.

According to an embodiment, said at least one compound is selected from the group consisting of 2-linoleoyl-glycerol (2-Lino-Gl), 2-palmitoyl-glycerol (2-Palm-Gl), N-palmitoylethanolamide (PEA), N-oleoyl ethanolamine (OEA) and combination thereof.

According to an embodiment, said treating induces the production of neurotransmitters and/or neuromodulators in the enteric nervous system.

According to an embodiment, said condition and/or symptom is selected from the group consisting of visceral sensation, visceral pain, visceral motility disorder, gastrointestinal motility disorder, inflammation, metabolic disorder, insulin resistance and combinations thereof.

According to an embodiment, said microorganism comprises a probiotic microorganism.

According to an embodiment, said microorganism is aerobic.

According to an embodiment, said microorganism is anaerobic.

According to an embodiment, said microorganism comprises a strain selected from the group consisting of Lactobacillus, Bifidobacterium, Prevotella, Bacteroides and combinations thereof.

According to an embodiment, said microorganism is selected from the group consisting of Lactobacillus rhamnosus, Lactobacillus crispatus, Lactobacillus iners, Lactobacillus gasseri, Lactobacillus jensenii, Lactobacillus acidophilus, Lactobacillus plantarum, Lactobacillus vaginalis, Lactobacillus casei, Lactobacillus reuteri, Lactobacillus salivarius, Lactobacillus Bifidobacterium Lactis, Streptococcus thermophilus, Bifidobacterium longum, Bifidobacterium breve, Bifidobacterium infantis, Lactobacillus helveticus, Lactobacillus rhamnosus, Clostridium, Akkermansia, Bacteroides, Parabacteriodes, Actinobacteria, Proteobacteria E. coli, Enterococcus, Prevotella, Corprococcus, and Veilonellaceae, Bacteroides. fragilis and combinations thereof.

According to an embodiment, said method further comprises feeding said animal a high-fat diet.

According to an embodiment, said method further comprises feeding said animal a low-fat diet.

According to an embodiment, administering said microorganism comprises feeding a food comprising a therapeutically effective amount of said microorganism.

According to an embodiment, said administering of said microorganism is conducted prior to or simultaneously with said administering said at least one compound.

According to an embodiment wherein said microorganism is administered simultaneously with said at least one compound, said microorganism and said at least one compound are provided in the same dosage form. According to an embodiment wherein said microorganism is administered simultaneously with said at least one compound, said microorganism and said at least one compound are provided in separate dosage form.

According to an embodiment, the method comprises multiple administrations of said microorganism and/or said prebiotic and multiple administrations of said at least one compound. According to an embodiment, the method comprises administration of said microorganism and/or said prebiotic four times per day, three times per day, twice per day, once per day, three times per week, twice per week, once per week, twice per month, once per month. According to an embodiment, the method comprises administration of said at least one compound four times per day, three times per day, twice per day, once per day, three times per week, twice per week, once per week, twice per month, once per month.

According to an embodiment, the amount of said at least one at least one compound administered with said microorganism is less than the amount of said at least one at least one compound which would be administered in the absence of said microorganism in order to achieve a same therapeutic effect. According to an embodiment, the amount of said at least one at least one compound administered with said microorganism is about 90%, about 80%, about 75%, about 70%, about 60%, about 50%, about 40%, about 30%, about 25%, about 20%, about 10% or even about 5% of that which would be administered in the absence of said microorganism in order to achieve a same therapeutic effect.

According to a further aspect of some embodiments of the present invention, there is provided a composition comprising

(i) a therapeutically effective amount of at least one microorganism and/or of a prebiotic thereof, wherein said microorganism is capable of modulating an effect of at least one element of the endocannabinoid system, and
(ii) a therapeutically effective amount of at least one compound capable of modulating an effect of at least one element of the endocannabinoid system.

According to an embodiment, said modulating the effect of said at least one element is a direct modulation.

According to an embodiment, said modulating the effect of said at least one element is an indirect modulation.

According to an embodiment, said at least one microorganism is provided in a dried form. According to one such embodiment, the microorganism is provided in a freeze-dried form.

According to an embodiment, said element is selected from the group consisting of endocannabinoid receptors, endocannabinoids, endocannabinoids synthesizing enzymes, endocannabinoids degrading enzymes and combinations thereof.

According to an embodiment, the at least one compound comprises at least one compound d, at least two compound s, at least three, at least four or at least five compounds. According to an embodiment, the content of each compound in the composition is at least 10 parts per million (ppm).

According to some embodiments, the at least one compound is a cannabinoid. As known in the art, cannabinoids have an acid form and a non-acid form (which is also referred to as decarboxylated form, since it can be generated by decarboxylating the acid form). The acid form is indicated herein by the letter (a) at the end of the cannabinoid acronym, e.g. tetrahydrocannabiniolic acid is indicated as THCa, while the decarboxylated form is THC. According to an embodiment, the cannabinoids are selected from the group consisting of tetrahydrocannabiniol in acid or decarboxylated form (THCa or THC, respectively), cannabidiol in acid or decarboxylated form (CBDa or CBD, respectively), cannabigerol in acid or decarboxylated form (CBGa or CBG, respectively), cannabichromene in acid or decarboxylated form (CBCa or CBC, respectively) tetrahydrocannabivarin in acid or decarboxylated form (THCVa or THCV, respectively), Cannabidivarin in acid or decarboxylated form (CBDVa or CBDV respectively) and cannabinol in acid or decarboxylated form (CBNa or CBN, respectively), Cannabicyclol in acid or decarboxylated form (CBLa or CBL, respectively).

According to an embodiment, at least one of the cannabinoids is in acid form. According to an embodiment, at least one of the cannabinoid is at least partially in decarboxylated form. According to an embodiment, at least 50% of the cannabinoid is in decarboxylated form, at least 60%, at least 70%, at least 80% or at least 90%.

According to an embodiment, the at least one cannabinoid comprises THC and/or THCa. According to an embodiment, the at least one cannabinoid comprises CBD and/or CBDa. According to an embodiment, the at least one cannabinoid comprises THC and/or THCa at a content of less than 1%, less than 0.8%, less than 0.6%, less than 0.4% or less than 0.2%. According to an embodiment, the at least one cannabinoid comprises both CBD and/or CBDa and THC and/or THCa and the weight/weight ratio between CBD and/or CBDa and THC and/or THCa ((CBD+CBDa)/(THC+THCa)) is at least 10, at least 15, at least 20, at least 25 or at least 30. According to an embodiment, the at least one cannabinoid comprises CBG and/or CBGa. According to an embodiment, the at least one cannabinoid comprises CBN and/or CBNa. According to an embodiment, the at least one cannabinoid comprises CBC and/or CBCa. According to an embodiment, the at least one cannabinoid comprises CBL and/or CBLa. According to an embodiment, the at least one cannabinoid comprises THCV and/or THCVa. According to an embodiment, the at least one cannabinoid comprises CBDV and/or CBDVa.

According to an embodiment, said cannabinoid is a phytocannabinoid and/or a synthetic cannabinoid.

According to an embodiment, said cannabinoid is selected from the group consisting of CBD, CBDA, CBDV, THCV, THC, THCA, CBG, CBGV, CBC, CBN and combination thereof.

According to an embodiment, said at least one compound is not a cannabinoid.

According to an embodiment, wherein said at least one compound is a terpene. According to one such embodiment, said terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, geranyl acetate, camphor, menthol, iso-menthone, neral, gerial, viridiflorol, germacrene, thymol, Menth-2-en-1-ol, farensol, carotol, myrtenol, citral and combination thereof.

According to an embodiment, said at least one compound is selected from the group consisting of 2-linoleoyl-glycerol (2-Lino-Gl), 2-palmitoyl-glycerol (2-Palm-Gl), N-palmitoylethanolamide (PEA), N-oleoyl ethanolamine (OEA) and combination thereof.

According to an embodiment, said composition induces the production of neurotransmitters and/or neuromodulators in the enteric nervous system.

According to an embodiment, the composition as disclosed herein is for use in treating a condition selected from the group consisting of visceral sensation, visceral pain, visceral motility disorder, gastrointestinal motility disorder, inflammation, metabolic disorder, insulin resistance and combinations thereof.

According to an embodiment, said microorganism comprises a probiotic.

According to an embodiment, said microorganism is aerobic.

According to an embodiment, said microorganism is anaerobic.

According to an embodiment, said microorganism comprises a strain selected from the group consisting of Lactobacillus, Bifidobacterium, Prevotella, Bacteroides and combinations thereof.

According to an embodiment, said microorganism is selected from the group consisting of Lactobacillus rhamnosus, Lactobacillus crispatus, Lactobacillus iners, Lactobacillus gasseri, Lactobacillus jensenii, Lactobacillus acidophilus, Lactobacillus plantarum, Lactobacillus vaginalis, Lactobacillus casei, Lactobacillus reuteri, Lactobacillus salivarius, Lactobacillus Bifidobacterium Lactis, Streptococcus thermophilus, Bifidobacterium longum, Bifidobacterium breve, Bifidobacterium infantis, Lactobacillus helveticus, Lactobacillus rhamnosus, Clostridium, Akkermansia, Bacteroides, Parabacteriodes, Actinobacteria, Proteobacteria E. coli, Enterococcus, Prevotella, Corprococcus, and Veilonellaceae, Bacteroides. fragilis and combinations thereof.

According to an embodiment, the composition further comprises a carrier. According to an embodiment, the composition comprises at least 2% by weight carrier, at least 3%, at least 5%, at least 10%, at least 15%, at least 20%, at least 25%, at least 30%, at least 35% or at least 40%, at least 50%, at least 60%, at least 70%, or at least 80% by weight carrier. Any compound other than cannabinoids and terpenes is a suitable carrier. According to an embodiment, the carrier is selected from the group consisting of vegetable oils, e.g. coconut oil, olive oil or sesame oil, pharmaceutical excipients, honey, bees wax, cellulose and combinations thereof. As used herein, the term “cellulose” refers to cellulose, hemicellulose and their combinations.

According to an embodiment, the composition comprises at least one terpene. The term “terpene”, as used herein, refers to both terpenes and terpenoids.

According to an embodiment, the at least one terpene, is selected from the group consisting of pinene, limonene, linalool, caryophyllene, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, geranyl acetate, camphor, menthol, iso-menthone, neral, gerial, viridiflorol, germacrene, thymol, Menth-2-en-1-ol, farensol, carotol, myrtenol isomers thereof and combinations thereof.

According to an embodiment, the composition comprises at least one flavonoid. According to an embodiment, the composition comprises at least two, at least three, at least four, or at least five flavonoids. According to an embodiment, the composition comprises at least one of bergamottin, apigenin, amentoflavone, quercetin and piperine.

According to an embodiment, the composition further comprises an additive selected from the group consisting of antioxidants, emulsifiers and texturizers vegetable oils, plant extracts, honey, pharmaceutical excipients, sucrose, glucose and fructose, pharmaceutical excipients and combinations thereof. According to an embodiment, the composition comprises a surfactant selected from the group consisting of phospholipids, glycerides, glycolipids and combinations thereof. According to an embodiment, the composition further comprises a food-approved texturizer. According to an embodiment, the composition further comprises at least 10 ppm ethanol. According to an embodiment, the composition further comprises at least one of vitamin C, vitamin E, polyunsaturated fatty acids, beeswax and coconut oil. According to an embodiment, the composition further comprises a sweetener.

According to an embodiment, said conditions and/or symptoms associated with autism and specific compositions associated therewith are as disclosed in PCT/IB2020/050635, which is incorporated by reference as if fully set out herein.

According to an embodiment, said composition is provided in a form selected from the group consisting of medical patches, stickers, topicals, creams, varnishes, sprays, edibles, beverages, suppositories, nasal preparations, inhalation mask, sub-lingual tabs, lozenges, chewing gums, preparations containing micro and/or nano-emulsions, preparations containing micro and/or nano-particles and combinations thereof.

Thus, the scope of the invention shall include all modifications and variations that may fall within the scope of the attached claims. Other embodiments of the invention will be apparent to those skilled in the art from consideration of the specification and practice of the invention disclosed herein. It is intended that the specification and examples be considered as exemplary only, with a true scope and spirit of the invention being indicated by the claimed embodiments.

### EXAMPLES

| Form | Microorgansism | Compound | Therapeutic effect |
|---|---|---|---|
| Sublingual oil | Lactobacillus rhamnosus | CBD | Motion sickness |
| Tablet | Streptococcus thermophilus | CBD, Limonene | Anorexia |
| Gel capsule | Bifidobacterium longum + | CBD, α-pinene | Parkinson's disease |
| | Lactobacillus casei | | |
| Tablet | Bifidobacterium breve + | CBD, Gingerol + | Irritable bowel disease |
| | Lactobacillus gasseri | caryophyllene | |
| Tablet | Bifidobacterium breve | THC, CBD | Migraine |
| Tablet | Lactobacillus jensenii | THC, CBD, α- | Multiple sclerosis |
| | | pinene, myrcene, | |
| | | humulene | |
| Sublingual oil | Bacteroide fragilis + | THC, CBD, α- | Fibromyalgia |
| | Clostridium | pinene, | |
| Sublingual oil | Streptococcus thermophilus | THC | Post-traumatic stress |
| | | | disorder |
| Tablet | Bifidobacterium breve + | THC | Diabetes |
| | Lactobacillus rhamnosus | | |
| Gel capsule | Lactobacillus helveticus | THC Pinene | Affective disorder |
| Sublingual oil | Lactobacillus rhamnosus | 2-Lino-GI | Visceral pain |
| Tablet | Streptococcus thermophilus | Limonene OEA | Gastrointestinal motility |
| Gel capsule | Bifidobacterium longum + | Linalool | Parkinson's disease |
| | Lactobacillus casei | | |
| Tablet | Bifidobacterium breve + | Gingerol, eugenol | Irritable bowel disease |
| | Lactobacillus gasseri | | |
| Tablet | Bifidobacterium breve | PEA | Migraine |
| Gel capsule | Lactobacillus jensenii | α-pinene, myrcene, | Multiple sclerosis |
| | | humulene | |
| Sublingual oil | Bacteroide fragilis + | α-pinene, | Fibromyalgia |
| | Clostridium | | |
| Sublingual oil | Streptococcus thermophilus | 2-Palm-GI | Inflammation |
| Tablet | Bifidobacterium breve + | PEA, caryophyllene | Diabetes |
| | Lactobacillus rhamnosus | | |
| Gel capsule | Lactobacillus helveticus | Pinene | Affective disorders |

What is claimed is:

1. A method of treating a condition and/or a symptom associated with an effect of at least one element of the endocannabinoid system in an animal, the method comprising administering to said animal (i) a therapeutically effective amount of at least one microorganism and/or of a prebiotic thereof, wherein said microorganism is capable of modulating an effect of at least one element of the endocannabinoid system of said animal; and (ii) a therapeutically effective amount of at least one compound capable of modulating an effect of at least one element of the endocannabinoid system of said animal.

2. The method of claim 1, wherein said microorganism and said at least one compound are capable of modulating a same said at least one element of said endocannabinoid system of said animal.

3. The method of claim 1, wherein said microorganism is capable of modulating a first said element of said endocannabinoid system and said at least one compound is capable of modulating a second element of said endocannabinoid system, wherein said first element is different from said second element.

4. The method of claim 1, wherein said condition and/or symptom is associated with at least one selected from the group consisting of the gastrointestinal tract, adipose tissue, and the immune system.

5. The method of claim 1, wherein said condition and/or symptom is selected from the group consisting of migraine, fibromyalgia, irritable bowel diseases, diabetes, post-traumatic stress disorders, affective disorders, anorexia, motion sickness, multiple sclerosis, Parkinson disease, autism spectrum disorder, clinical deficiency of an endocannabinoid, visceral sensation, visceral pain, visceral motility disorder, gastrointestinal motility disorder, inflammation, metabolic disorder, insulin resistance and combinations thereof.

6. The method of claim 1, wherein said at least one element of the endocannabinoid system is selected from the group consisting of endocannabinoid receptors, endocannabinoids, endocannabinoids synthesizing enzymes, endocannabinoids degrading enzymes, and combinations thereof.

7. The method of claim 1, wherein said at least one microorganism modulates the expression of at least one selected from the group consisting of cannabinoid type 1 (CB1) receptors, cannabinoid type 2 (CB2) receptors, transient receptor potential (TRP) channels, Peroxisome proliferator-activated receptors (PPARs), anandamide, 2-Arachidonoylglycerol (2AG), fatty acid amide hydrolase (FAAH), N-acyl phosphatidylethanolamine phospholipase D (NAPE-PLD), monoacylglycerol lipase (MAGL), diacylglycerol (DAG) lipases or combinations thereof.

8. The method of claim 1, wherein said at least one compound is selected from the group consisting of 2-linoleoyl-glycerol (2-Lino-Gl), 2-palmitoyl-glycerol (2-Palm-Gl), N-palmitoylethanolamide (PEA), N-oleoyl ethanolamine (OEA) and combination thereof.

9. The method of claim 1, wherein said treating induces the production of neurotransmitters and/or neuromodulators in the enteric nervous system.

10. The method of claim 1, wherein said at least one microorganism comprises a strain selected from the group consisting of Lactobacillus, Bifidobacterium, Prevotella, Bacteroides and combinations thereof.

11. The method of claim 1, wherein said administering said at least one microorganism comprises feeding said animal a food comprising a therapeutically effective amount of said microorganism and/or said prebiotic.

12. The method of claim 1, wherein said administering said at least one microorganism is conducted prior to or simultaneously with said administering said at least one compound.

13. The method of claim 1, comprising multiple administrations of said at least one microorganism and/or said prebiotic and multiple administrations of said at least one compound.

14. The method of claim 1, wherein said at least one microorganism and/or said prebiotic and said at least one compound are provided as a kit.

15. A composition comprising
(i) a therapeutically effective amount of at least one microorganism and/or of a prebiotic thereof, wherein said at least one microorganism is capable of modulating an effect of at least one element of the endocannabinoid system, and
(ii) a therapeutically effective amount of at least one compound capable of modulating an effect of at least one element of the endocannabinoid system.

16. The composition of claim 15, wherein said compound is an allocentric modulator of a gastrointestinal tract endocannabinoid receptor.

17. An animal food comprising the composition of claim 15 and at least one food ingredient.
